(19) United States
(12) Patent Application Publication
(10) Pub. No.: US 2008/0293587 A1
(43) Pub. Date: Nov. 27, 2008

(54) Method of Screening a Biological Target for Weak Interactions Using Weak Affinity Chromatography

(75) Inventors: Sten Ohlson, Malmo (SE); Siamak Shoravi, Kalmar (SE); Tomas Fex, Lund (SE); Roland Isaksson, Lund (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(73) Assignee: Transientic Interactions AB

(21) Appl. No.: 12/090,784

(22) PCT Filed: Oct. 25, 2006
(86) PCT No.: PCT/SE2006/050419

§ 371 Date: Apr. 18, 2008

Related U.S. Application Data
(60) Provisional application No. 60/730,808, filed on Oct. 27, 2005.

(51) Int. Cl. C40B 30/04

(52) U.S. Cl. 506/9

(57) ABSTRACT

The present invention relates to a method of screening a biological target for transient weak interactions between the target and a library of ligands. The method includes the provision of a composition comprising a biological target and the provision of a plurality of stationary phases from such a composition. A plurality of ligand compositions is transported to the stationary phases to establish contacts between the ligands and the biological targets. Zonal retardation information are collected for each ligand, downstream of the stationary phases in order to select ligands with dissociation constants (Kd) in the range of 0.01 to 10 mM, exhibiting weak affinity to the target.

### BACKGROUND OF INVENTION

The notion that many biological interactions are based on transient binding (dissociation constants (K_{d}) in the range of 10 mM to 0.01 mM) is familiar, yet the implications for biological sciences have been realized only recently (Gabius, H.-J. & Gabius, S. (eds.) Glycosciences: Status and Perspectives (Chapman & Hall, Weinheim, 1997)). An important area of biological sciences is drug design where the traditional ‘lock and key’ view of binding has prevailed and drug candidates are usually selected on their merits as being tight binders. However, the rationale that transient interactions are of importance for drug discovery is slowly gaining acceptance. These interactions may relate not only to the desired target interaction, but also to unwanted interactions creating toxicity problems or to interactions with drug carriers involved in absorption and/or excretion (Steffansen, B. et al. Intestinal Solute Carriers: An Overview of Trends and Strategies for Improving Oral Drug Absorption. Eur. J. Pharm. Sciences 21, 3-16 (2004)). Here we demonstrate, in a high-throughput screening format, affinity selection of weak binders to a model target of albumin by zonal retardation chromatography (Steffansen, B. et al. Intestinal Solute Carriers: An Overview of Trends and Strategies for Improving Oral Drug Absorption. Eur. J. Pharm. Sciences 21, 3-16 (2004)). It is perceived that this approach can define the ‘transient drug’ as a complement to current drug discovery procedures.

Transient biological interactions form the essence of the interactome and occur constantly inside the cells, on cell surfaces or in the extra cellular matrix. They are ‘dynamic’, with dissociation rate constants (k_{d})>0.1 s⁻¹ and are governed by the local concentrations of the interacting biological pair. They can act in parallel, such as the interactions of the stacked bases of DNA, or in a serial fashion such as the action of small molecule substrates or inhibitors upon an enzyme. Biological macromolecules undergo a number of transient interactions through their component amino acids, carbohydrates, lipids or nucleotides. These macromolecules form assemblies expressed as cellular organelles which are involved in a complex interplay of weak interactions forming the network of the biological entity.

As technologies are evolving to study and characterize transient interactions based on chromatography (Strandh, M., Andersson, H. & Ohlson, S. in Methods in Molecular Biology (eds. Bailon, P., Ehrlich, G. K., Fung, W. J. & Berthold, W.) 7-24 (Humana Press, Inc, Totowa, N.J., 2000)), electrophoresis (Nilsson, M. et al. Determination of Protein-Ligand Affinity Constants from Direct Migration Time in Capillary Electrophoresis. Electrophoresis 25, 1829-1836 (2004)), surface plasmon resonance (SPR) biosensor (MacKenzie, C. R. et al. Analysis by Surface Plasmon Resonance of the Influence of Valence on the Ligand Binding Affinity and Kinetics of an Anti-Carbohydrate Antibody. The Journal of Biological Chemistry 271, 1527-1533 (1996)), fluorescence spectroscopy (Engstrom, H. A., Andersson, P. O. & Ohlson, S. Analysis of the Specificity and Thermodynamics of the Interaction between Low Affinity Antibodies and Carbohydrate Antigens using Fluorescence Spectroscopy. J Immunol Methods 297, 203-211 (2005)), nuclear magnetic resonance (NMR) (Pellechia, M., Sem, D. S. & Wuthrich, K. NMR in Drug Discovery. Nature Reviews in Drug Discovery 1, 211-219 (2002)), calorimetry (Plotnikov, V. et al. An Autosampling Differential Scanning Calorimeter Instrument for Studying Molecular Interactions. Assay Drug Dev. Technol. 1, 83-90 (2002)) and chemiluminescence (Causey, L. D. & Dwyer, D. S. Detection of Low Affinity Interactions Between Peptides and Heat Shock Proteins by Chemiluminescence of Enhanced Avidity Reactions (CLEAR). Nature Biotechnology 14, 348-351 (1996)), we are now gradually unraveling the nature of transient binding in various areas such as bacterial and viral interactions (Mammen, M., Choi, S.-K. & Whitesides, G. M. Polyvalent interactions in biological systems: Implications for design and use of multivalent ligands and inhibitors. Angew. Chem. Int. Ed. 37, 2754-2794 (1998)), cellular interactions (Dustin, M. L. et al. Low Affinity Interaction of Human or Rat T Cell Adhesion Molecule CD2 with Its Ligand Aligns Adhering Membranes to Achieve High Physiological Affinity. The Journal of Biological Chemistry 272, 30889-30898 (1997)), protein-protein (peptide) interactions (Karjalainen, K. High-Sensitivity, Low Affinity-Paradox of T-Cell Receptor Recognition. Current Opinion in Immunology 6, 9-12 (1994); Beeson, C. et al. Early Biochemical Signals Arise from Low Affinity TCR-Ligand Reactions as the Cell-Cell Interface. J. Exp. Med 184, 777-782 (1996); and Garcia, C. D., DeGail, J. H., Wilson, W. W. & Henry, C. S. Measuring Protein Interactions by Microchip Self-Interaction Chromatography. Biotechnol. Prog. 19, 1006-1010 (2003)), protein-carbohydrate interactions (Zopf, D. & Roth, S. Oligosaccharide Anti-infective Agents. Lancet 347, 1017-1021 (1996); and Elgavish, S. & Shaanan, B. Lectin-Carbohydrate interactions: Different Folds, Common Recognition Principles. TIBS 22, 462-467 (1997)) and carbohydrate-carbohydrate interactions (Fuente, J. M. d. l. & Penadés, S. Understanding Carbohydrate-Carbohydrate Interactions by means of Glyconanotechnology. Glycoconjugate Journal 21, 149-163 (2004)).

The very nature of transient biological interactions makes them attractive in the laboratory for at least two major lines of investigation. First, as demonstrated in recent years, they can be used in diagnostic applications to continuously monitor fluctuating amounts of biomolecules in real-time (Ohlson, S., Jungar, C., Strandh, M. & Mandenius, C.-F. Continuous Weak-affinity Immunosensing. Trends in Biotechnology 18, 49-52 (2000)) or to develop more specific recognition molecules to targets where traditional approaches of high-affinity ligands may not offer a viable solution (Regenmortel, M. H. V. V. From absolute to exquisite specificity. Reflections on the fuzzy nature of species, specificity and antigenic sites. J. Immunol. Methods 216, 37-48 (1998); and Leickt, L., Grubb, A. & Ohlson, S. Development of Monoclonal Antibodies against creatine kinase CKMB2. Scand J Clin Lab Invest 62, 423-430 (2002)). Second, due to the transient nature of many interactions in a biological entity, it can be envisaged that synthetic drugs can be similar in terms of binding behaviour to their natural counterparts. Such ‘transient’ drugs can be attractive for a number of reasons. For example, they can, either in a monovalent or a polyvalent format, be designed with specificity superior to that of the traditional high affinity drug. Tolerance or severe side effects of drug action may be minimized with a weakly binding drug e.g. in its binding to cytochrome p450 proteins. Drug absorption can be increased through weak interactions with specific uptake mechanisms. Also the active concentration may be an efficient tool to govern the biological effect of the drug.

Furthermore, in the race for new drug molecules, there is increased interest to search compound libraries with small molecules which can subsequently be optimized by increasing size and introducing additional functionalities. Such fragment based approaches (Erlanson, D. A., McDowell, R. S. & O'Brien, T. Fragment-Based Drug Discovery. Journal of Medicinal Chemistry 47, 3463-3482 (2004)), which identify small molecules that bind with low affinity, are being used increasingly in drug discovery. Weak-binding drug candidate molecules in practice often are difficult to screen for. There are methods available based on NMR, mass spectrometry and X-ray crystallography but these are not designed to detect weak binders on a high throughput basis, where thousands of samples can be screened per day. In addition ‘virtual screening’ methods have also been applied with limited success, mainly with rigid inflexible fragments (Erickson, J. A., Jalaie, M., Robertson, D. H., Lewis, R. A. & Vieth, M. Lessons in Molecular Recognition: The Effects of Ligand and Protein Flexibility on Docking Accuracy. Journal of Medicinal Chemistry 47, 45-55 (2004)).

In recent years high throughput screening (HTS) of compound libraries has become an important tool for identification of potential drug candidates (Sundberg, S. A. High-Throughput and Ultra-High-Throughput Screening: Solution- and Cell-Based Approaches. Current Opinion in Biotechnology 11, 47-53 (2000); and Williams, G. P. Advances in High Throughput Screening. Drug Discovery Today 9, 515-516 (2004)). The HTS technology has evolved rapidly with the development of automated and miniaturized equipment that now can handle several hundred thousand samples. Standard-plate binding assay techniques are typically used with fluorescence, radioactivity, or optical absorbance as the detection platform. One important restriction with these techniques is that they can only measure strong interactions (a typical assay concentration is 10 μM) because transient drug binders are washed away in the assay procedure. Another limitation is that they only offer evidence of binding where identification of the binding partner has to be implemented.

Affinity chromatography has developed into a powerful tool mainly for the purification of proteins. In addition frontal affinity chromatography in combination with mass spectrometry has been used for drug discovery applications (Chan, N. W. C., Lewis, D. F., Rosner, P. J., Kelly, M. A. & Schriemer, D. C. Frontal Affinity Chromatography-Mass Spectrometry Assay Technology for Multiple Stages of Drug Discovery: Applications of a Chromatographic Biosensor. Anal Chem 319, 1-12 (2003)) for detecting high-affinity binders. Now, with the introduction of weak affinity chromatography (WAC) (Ohlson, S., Lundblad, A. & Zopf, D. Novel Approach to Affinity Chromatography Using “Weak” Monoclonal Antibodies. Analytical Biochemistry 169, 204-208 (1988)) for zonal retardation, high performance methods are emerging which provide information about affinity and kinetics of weak interactions with biomolecules, wherein chromatographic separation is usually carried out under mild isocratic conditions, which could be physiologically relevant. In order to efficiently identify candidates exhibiting weak affinity, there is a need for a methodology that supports rapid screening and selection of potential drug molecules. The present invention aims at providing such a method.

### DESCRIPTION OF THE INVENTION

The present invention relates to a method of screening a biological target for transient weak interactions between the target and a library of ligands. The inventive method comprises the steps of providing a composition of a biological target; providing a plurality of stationary phases from said composition; transporting a plurality of ligand compositions to said stationary phases, thereby establishing contacts between said ligands and said biological targets; collecting, downstream of said stationary phases, zonal retardation information for each ligand; and finally selecting ligands exhibiting weak affinity to said target, wherein said ligands have dissociation constants (K_{d}) in the range of about 0.01 to about 10 mM. The selected ligands having transient bindings to the biological target may undergo further studies regarding their binding behaviour, for example with NMR analysis, with the purpose of identifying one or several lead compound(s). The method typically involves a detection step, wherein ligands arriving from said stationary phases during a time period sufficient to discriminate between different ligand affinities and for this purpose collecting zonal retardation information, most importantly retention time and bandwidth to estimate the affinity and the dynamics of each ligand/target interaction. The nature of the detection process is not critical for the invention and is rather determined with respect to the chemistry of the ligand library.

According to one aspect, the biological target is provided an immobilized composition, for example the target is immobilized to a solid support. The person skilled in this technology knows a number of suitable support materials and means to adsorb or chemically link biological molecules thereto. In one example, the immobilized composition may be used with chromatography columns or multi-well systems and means for elution with a mobile phase. In a specific example, a proteinaceous target can be immobilized to functionalized silica particles with conventional methods and packaged into a plurality of chromatography wells, whereupon ligand compositions are injected in each well before elution with mobile phase and collection of fractions for detection.

According to another aspect, stationary phases including a biological target can be formed in a in a plurality of miniaturized channels in a solid support, such as thin plate, a chip or a disc. The solid support may have a first zone for receiving a plurality of ligand compositions for transportation to said biological target and a detection zone downstream of said first zone. Suitably, the transportation in the solid support takes place by capillary forces and/or centrifugal forces when ligands travel from the first zone to the detection zone.

In an example on how the inventive method can practically be realized, a system is formed which includes a multi-well plate with immobilized biological target in each of the wells. The system further comprises means for supplying compositions of the ligands to each well and a series of matching collector plates for collecting fractions of eluted mobile phase and subsequent detection. The system may further comprise means to assist with supplementation, transportation and elution of the mobile phase. The following part of the specification provides more detailed and illustrative examples on how conduct the invention and the skilled person may be able to deduce a number of alternatives that still will fall under the appended claims.

### DETAILED AND EXEMPLIFYING DESCRIPTION OF THE INVENTION

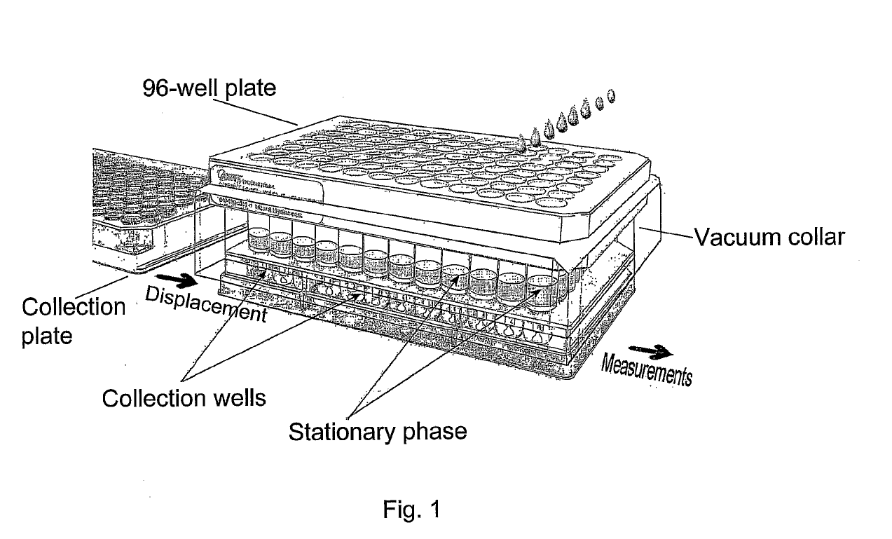

FIG. 1: Schematic set-up of zonal affinity separation screening in a 96-well format.

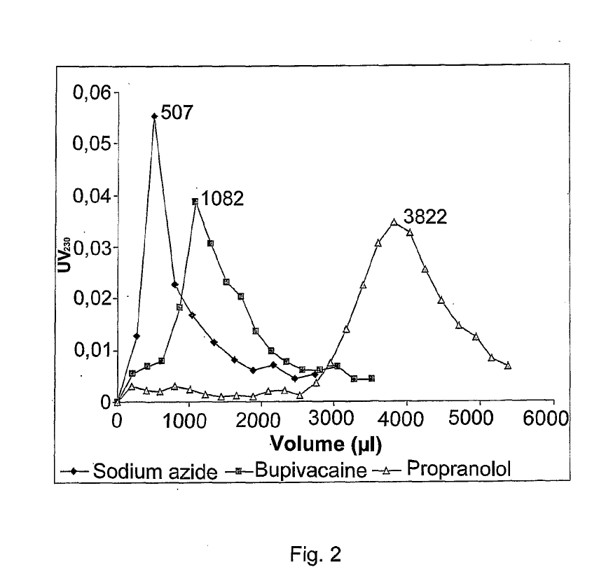

FIG. 2: Zonal weak affinity chromatography in a well. Bupivacain (1 mM) and propranolol (0.25 mM) were retarded as indicated by their peak apex. Sodium azide (0.5 mM) was a marker of void volume (non-retarded substance).

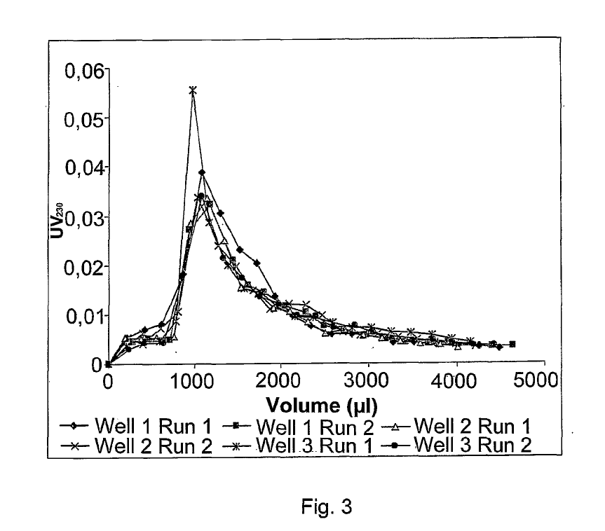

FIG. 3: Well-to-well reproducibility of zonal weak affinity chromatography of bupivacain (1 mM). Bupivacain was retarded in three different wells and chromatography was repeated once in each well.

In this model study we have looked at the weak interactions between some known drugs and bovine serum albumin (BSA). Proteins like orosomucoid, bovine albumin and human albumin have successfully been utilized as selectors to determine the enantiomeric composition of a large number of drugs. Here the affinity between drug and protein typically fall in the range of a transient interaction i.e. K_{d}-values of 10⁻¹-10⁻⁵ M. It is well-known from earlier studies that BSA is an excellent model protein and shows variable binding affinity as well as (enantio)selectivity to a large number of drugs (Haginaka, J. Protein-based Chiral Stationary Phases for High-performance Liquid Chromatography Enantioseparations. J of Chromatography A 906, 253-273 (2001)). Even with the somewhat basic experimental set-up in a 96-well format (FIG. 1), our results demonstrate (FIG. 2) that small selectivity differences can be detected in spite of the short retention times, indicating its potential use for HTS applications. Substances are slightly retarded through transient binding, probably with affinities of 0.1-1 mM, as a consequence of high active concentration of albumin binding sites (approximately 1 mM). Furthermore the separations show excellent reproducibility as can be seen in FIG. 3. The experiments also demonstrate the potential to run weak affinity separations in a parallel mode and in a multi-well plate format. With optimization of column packing techniques and liquid handling methods the relatively large volumes of the eluted zones (bands) will likely be reduced. The retention differences between propranolol, bupivacaine and sodium azide (giving the non-retained elution volume) are quite significant and in agreement with high performance liquid chromatography methods using corresponding commercial analytical columns.

The results presented here indicate the potential to use chromatography for screening of transient binding phenomena. By robotization and miniaturization of this analytical platform we anticipate that thousand of separations can be executed in one run in a matter of minutes. The results provide instant information about affinity and kinetics of relevant transient binders in terms of their retention time and band spreading. With the results above, we are convinced that the approach presented in this letter may offer a complementary tool that may open up alternative areas of drug research, looking for ‘transient drugs’ that bind dynamically to their targets. By implementing an HTS platform based on weak affinity chromatography, we will be able to explore this hypothesis in greater detail and to determine how this concept for drug discovery can be exploited

### Methods

### Preparation of BSA-Silica Material:

Diol microparticulate silica (10 m particles, pore size 300 A) was prepared according to standard methods (Ohlson, S., Lundblad, A. & Zopf, D. Novel Approach to Affinity Chromatography Using “Weak” Monoclonal Antibodies. Analytical Biochemistry 169, 204-208 (1988)) using γ-glycidoxipropyltrimethoxysilane as the silanization reagent. In a subsequent step, the diol phase was oxidized using periodic acid (H₅IO₆) to produce the aldehyde silica. To this support material, bovine serum albumin (BSA) was immobilized by reductive amination using sodiumcyanoborohydride to reduce the Schiff's base intermediate (BSA-diol-silica) (Ohlson, S., Lundblad, A. & Zopf, D. Novel Approach to Affinity Chromatography Using “Weak” Monoclonal Antibodies. Analytical Biochemistry 169, 204-208 (1988)). According to UV-analysis (280 nm) directly on gel, 130 mg BSA per g silica was immobilized corresponding to a yield of 80%.

### Preparation and Packing of Chromatography Wells:

200 mg of reference gel (diol-silica) and assay gel (BSA-diol-silica) were suspended in 1 ml methanol and packed into each well in a 96-well filtering system (Captiva® 96-well 10μ glassfiber filter plate from Ansys Technologies (Company), see FIG. 1) using vacuum applied to the wells. The gel in the wells was first washed with 5 ml MeOH and then with 5 ml 0.1 M sodium phosphate buffer pH 7.0. Filter discs were placed on top of the gel material in each well.

### Zonal Weak Affinity Chromatography in a 96-Well Plate Format: (See FIG. 1)

Wells containing gel materials were washed carefully with 0.05 M sodium phosphate buffer pH 7.0 using vacuum collar to remove non-bound BSA, contaminants and entrapped air. The excess of buffer was gently removed from the surface of the wells using pipette and 10 μl of test substances were injected centrally in each wells which were then flushed with mobile phase (0.05 M sodium phosphate buffer pH 7.0). Vacuum was applied to the collar and the mobile phase was flowed into a 96 well collection plate (Nunclon, Nalgene-Nunc) within 8-10 seconds. Vacuum was then reduced and the collection plate (approximately 0.2 ml in each well) was replaced with a new one. This fraction collection process was repeated with 20-75 plates. During this procedure the wells were eluted with mobile phase continuously. Caution was exercised to ensure that the level of buffer in each well did not fall below the surface level of gel. The eluates in the plates were transferred carefully by automatic pipettes and weighed to estimate the retention volume. All procedures were performed at room temperature (20° C.). Eluted substances were detected by UV-scans at 230 nm. Chromatograms were obtained by plotting UV absorbance versus fraction volume.

1. A method of screening a biological target for transient weak interactions between the target and a library of ligands comprising the steps of:
(i) providing a composition of a biological target;
(ii) providing a plurality of stationary phases from said composition;
(iii) transporting a plurality of ligand compositions to said stationary phases, thereby establishing contacts between said ligands and said biological targets;
(iv) collecting, downstream of said stationary phases, zonal retardation information for each ligand; and
(v) selecting ligands exhibiting weak affinity to said target, wherein said ligands have dissociation constants (K_{d}) in the range of 0.01 to 10 mM.

2. A method according to claim 1, wherein the zonal retardation information is selected among retention time and bandwidth.

3. A method according to claim 1, including quantitatively detecting said ligands in compositions arriving from said stationary phases during a time period sufficient to discriminate between different ligand affinities.

4. A method according to claim 1, comprising providing an immobilized composition of a biological target.

5. A method according to claim 4 comprising immobilizing the biological target to a solid support.

6. A method according to claim 4 comprising continuously eluting said stationary phase with a mobile phase.

7. A method according to claim 1, comprising providing a plurality of stationary phases with biological target in a plurality of miniaturized channels in a solid support.

8. A method according to claim 7, wherein said solid support comprises a first zone for receiving a plurality of ligand compositions for transportation to said biological target.

9. A method according to claim 8, wherein the solid support comprises a detection zone downstream of said first zone.

10. A method according to claim 9, wherein capillary forces and/or centrifugal forces serve for the transportations from first zone to the detection zone.

11. A method according to claim 2, comprising providing an immobilized composition of a biological target.

12. A method according to claim 3, comprising providing an immobilized composition of a biological target.

13. A method according to claim 5 comprising continuously eluting said stationary phase with a mobile phase.

14. A method according to claim 2, comprising providing a plurality of stationary phases with biological target in a plurality of miniaturized channels in a solid support.

15. A method according to claim 14, wherein said solid support comprises a first zone for receiving a plurality of ligand compositions for transportation to said biological target.

16. A method according to claim 15, wherein the solid support comprises a detection zone downstream of said first zone.

17. A method according to claim 16, wherein capillary forces and/or centrifugal forces serve for the transportations from first zone to the detection zone.

18. A method according to claim 3, comprising providing a plurality of stationary phases with biological target in a plurality of miniaturized channels in a solid support.

19. A method according to claim 18, wherein said solid support comprises a first zone for receiving a plurality of ligand compositions for transportation to said biological target.

20. A method according to claim 19, wherein the solid support comprises a detection zone downstream of said first zone.
